(19) United States
(12) Patent Application Publication
(10) Pub. No.: US 2009/0198165 A1
(43) Pub. Date: Aug. 6, 2009

(54) BRACE FOR ANKLE SUPPORT

(76) Inventor: Pang-Ching Chiang, Taipei (TW)

Correspondence Address: BURR & BROWN, PO BOX 7068, SYRACUSE, NY, 13261-7068, US

(21) Appl. No.: 12/025,283

(22) Filed: Feb. 4, 2008

(51) Int. Cl. A61F 5/01

(52) U.S. Cl. 602/27

(57) ABSTRACT

A brace has a shell formed within a contour that according to a person's ankle and a binding to fasten the shell onto the ankle. When the person is wearing an ankle support, the brace is mounted on an external surface of the ankle support and fastened by the binding. Such that the brace supports the ankle support and provides an extra constriction to the person's ankle to provide an extra effect of ankle immovability and impact cushion. The brace supports the ankle support via an inner surface evenly abuts the whole external surface of the ankle support. Thus, a pressure is evenly applied to the person's ankle and ordinary blood flow in the person's ankle portion is allowed so that the brace is comfortable to wear.

### BACKGROUND OF THE INVENTION

1. Field of the Invention

The present invention relates to a brace, and more particularly to a brace for an ankle support that is comfortable and provides improved immobilization to a person's ankle, thereby effectively preventing injuries occurring.

2. Description of Related Art

To prevent sports injuries and aid recovery, people wear various protective and supportive accessories to perform exercise, including: kneepads, knee supports, ankle supports, elbow pads, wrist pads and the like.

Aforesaid ankle supports are typically a resilient sock worn on an ankle to restrict lateral movement, iversion and eversion of the ankle and prevent ankle injuries.

However, after a long-term use, ankle supports become loose and are thus inadequate to provide protection. To resolve such problem, binding straps are further provided ankle support to adjustably tighten and loosen the support around the ankle. Each binding strap is attached to the ankle support, wound around the ankle support and has a fastener to attach the binding strap the ankle support to ensure adequate restriction of the ankle.

However, winding the binding strap ankle support too tightly may obstruct ordinary blood flow, thereby lengthening recover time, causing discomfort and prevent longitudinal movement required to partake in sports. Winding the binding strap too loosely does not provide adequate support.

To overcome these disadvantages, the present invention provides a brace for ankle support to obviate or mitigate the aforementioned problems.

### SUMMARY OF THE INVENTION

The main objective of the invention is to provide a brace to cover an ankle support in a comfortable manner and provides an extra constriction effect to a person's ankle, thereby effectively prevent sport injury occurrence.

To achieve the objective, the brace in accordance with present invention is formed within a contour that according to the person's ankle and has a binding.

When the person is wearing an ankle support, the brace is mounted on an external surface of the ankle support and fastened by the binding. Such that the brace supports the ankle support and provides an extra constriction to the person's ankle to provide an extra effect of ankle immovability and impact cushion.

Unlike conventional binding straps, the brace supports the ankle support via an inner surface evenly abuts the whole external surface of the ankle support. Thus, a pressure is evenly applied to the person's ankle and ordinary blood flow in the person's ankle portion is allowed so that the brace is comfortable to wear.

Other objectives, advantages and novel features of the invention will become more apparent from the following detailed description when taken in conjunction with the accompanying drawings.

### BRIEF DESCRIPTION OF THE DRAWINGS

FIG. 1 is a side view of a first embodiment of a brace for an ankle support in accordance with the present invention;

FIG. 2 is a perspective view of the brace in FIG. 1, in a first operational embodiment;

FIG. 3 is a perspective view of the brace in FIG. 1, in a second operational embodiment;

FIG. 4 is an operational perspective view of a second embodiment of a brace in accordance with the present invention; and

FIG. 5 is an operational perspective view of a third embodiment of a brace in accordance with the present invention.

### DETAILED DESCRIPTION OF THE PREFERRED EMBODIMENT

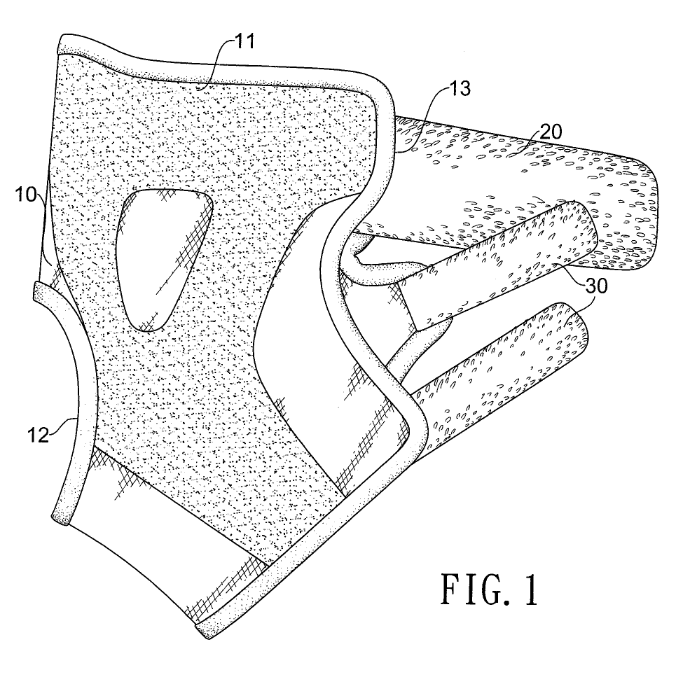

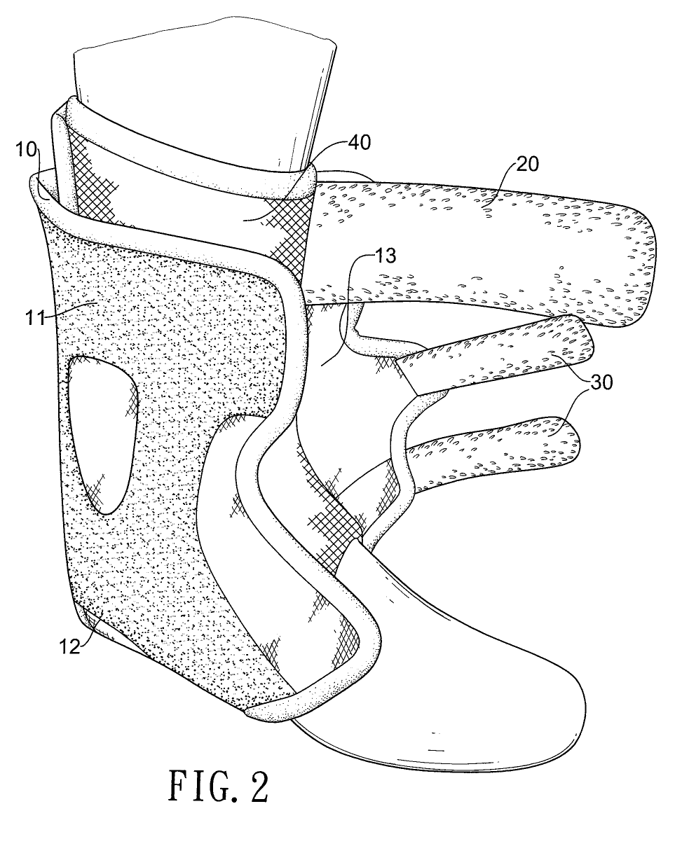

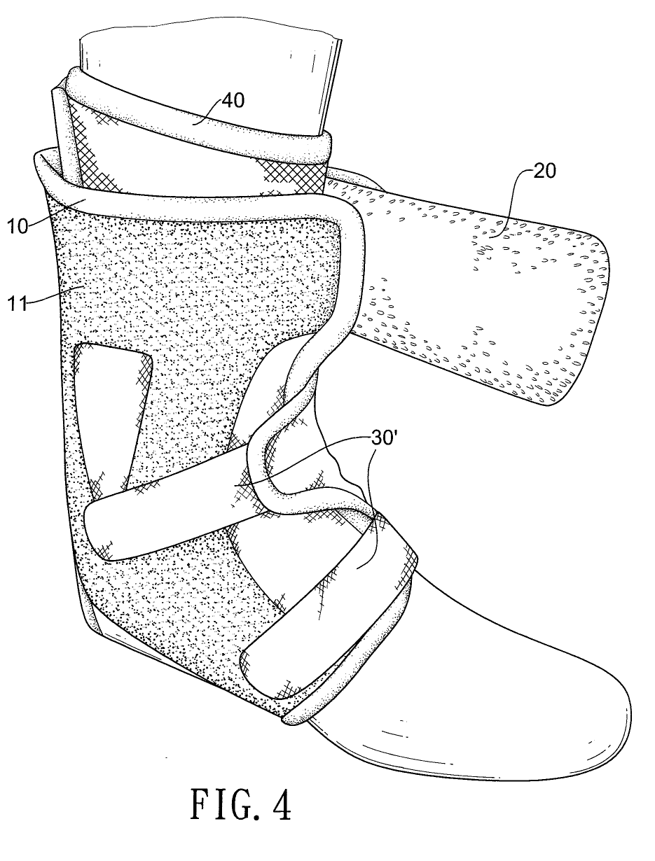

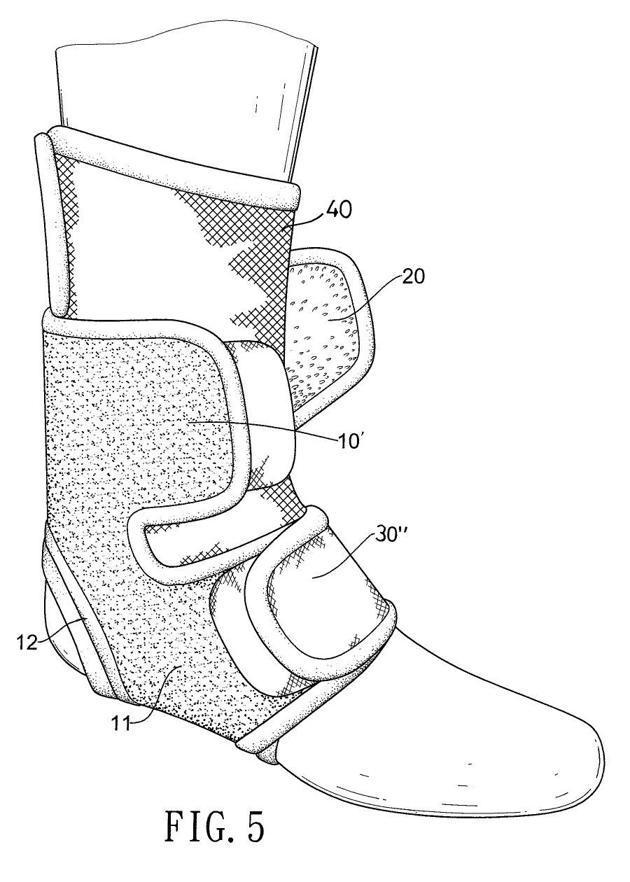

With reference to FIGS. 1, 2, 4 and 5, a brace for an ankle support in accordance with the present invention comprises a shell (10, 10′) and a binding.

The shell (10, 10′) corresponds to an ankle is rigid and has an external surface, an opening (13), two front edges, a rear, two side surfaces (11), and a heel opening (12). The external surface of the shell (10, 10′) may be half of a hook and loop fastener. The opening (13) allows the ankle to be easily inserted into the brace. The heel opening (12) is formed through the rear of the shell (10, 10′) corresponds to a heel and improves comfort.

The binding is attached securely adjacent to the front edge of the shell (10, 10′) and may comprise at least one strap or multiple laces. Each strap is securely attached to one front edge and selectively attached securely using a buckle or at least one hook and loop fastener to the other front edge to adjustably secure the strap around the shell (10, 10′). The laces are alternatively attached adjacent to the front edges of the shell (10, 10′), may be using protrudent hooks or through holes and are tied to each other.

When one strap is implemented, the strap is wound from one front edge above the ankle, over the front opening (13), behind the ankle, under a sole of the foot and adjustably attached adjacent to the other front edge. Multiple hook and loop fasteners may be implemented to ensure constant tension throughout the strap.

Preferably, the binding comprises at least one primary strap (20) and at least one secondary strap (30, 30′, 30″). Preferably, each strap has one hook and loop fastener.

The primary strap (20) is attached securely to one front edge, is wound over the front opening (13) and is adjustably attached adjacent to the other front edge above the ankle.

When one secondary strap (30″) is implemented, the secondary strap (30″) is as wide as the primary strap (20), is attached securely to one front edge and is wound over the front opening (13) and is adjustably attached adjacent to the other front edge, adjacent to the sole.

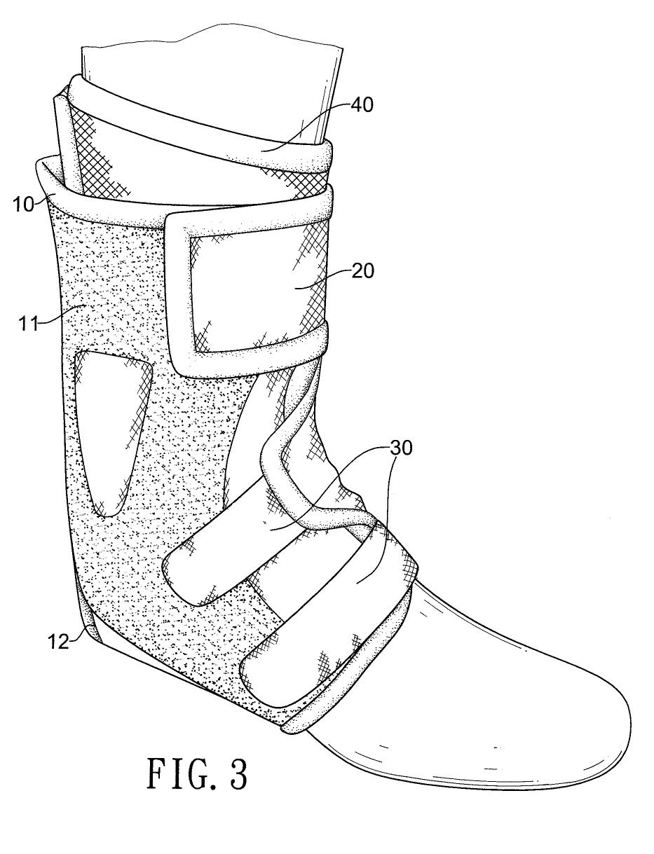

With further reference to FIG. 3, when two secondary straps (30, 30′) are implemented, the secondary straps (30, 30′) are narrower than the primary strap (20) are attached securely to one front edge, are wound over the front opening (13) and are adjustably attached adjacent to the other front edge, adjacent to the sole. The secondary straps (30) may be parallel to each other or the secondary straps (30′) may be divergent with respect to each other. When the person is wearing an ankle support (40) having an external surface, the brace is mounted around the external surface of the ankle support (40) and fastened by the binding. Therefore, the brace provides additional protection to the ankle support and restricts movement effectively to expedite recovery and prevent impact injuries.

Unlike conventional ankle supports, the brace applies even pressure around the ankle support to allow blood flow to be unaffected and raising comfort and further aiding recovery.

Even though numerous characteristics and advantages of the present invention have been set forth in the foregoing description together with details of the structure and function of the invention, the disclosure is illustrative only. Changes may be made in detail especially in matters of shape, size, and arrangement of parts within the principles of the invention to the full extent indicated by the broad general meaning of the terms in which the appended claims are expressed.

What is claimed is:

1. A brace comprising
a shell being hollow and having
a front;
a rear;
two side surfaces; and
an opening being defined throughout through the front of the shell; and
a binding including at least one primary strap and at least one secondary strap being arranged along the opening of the shell, and each of the at least one primary strap and the at least one secondary strap having
a proximal end being attached securely to one of the side surface of the shell; and
a distal end being detachably mounted to the other side surface of the shell.

2. The brace as claimed in claim 1, wherein the distal end of each one of the at least one secondary strap is detachably mounted to the side surface of the shell via hook and loop.

3. The brace as claimed in claim 1, wherein the binding includes one primary strap and two secondary straps.

4. The brace as claimed in claim 3, wherein the secondary straps are parallel to each other.

5. The brace as claimed in claim 3, wherein the secondary straps are divergent with respect to each other.

6. The brace as claimed in claim 1, wherein the binding includes one primary strap and one secondary strap with a same width.

7. The brace as claimed in claim 1, wherein the shell further has a heel opening being formed through the rear of the shell.
